(19) United States
(12) Patent Application Publication
(10) Pub. No.: US 2017/0014548 A1
(43) Pub. Date: Jan. 19, 2017

(54) MAGNESIUM/POLYMER COMPOSITE-CONTAINING SCAFFOLDS TO ENHANCE TISSUE REGENERATION

(71) Applicant: UNIVERSITY OF PITTSBURGH-OF THE COMMONWEALTH SYSTEM OF HIGHER EDUCATION, PITTSBURGH, PA (US)

(72) Inventors: Charles S. SFEIR, Pittsburgh, PA (US); Andrew J. BROWN, Pittsburgh, PA (US)

(73) Assignee: UNIVERSITY OF PITTSBURGH-OF THE COMMONWEALTH SYSTEM OF HIGHER EDUCATION, PITTSBURGH, PA (US)

(21) Appl. No.: 15/124,093

(22) PCT Filed: Mar. 13, 2015
(86) PCT No.: PCT/US2015/020338

§ 371 Date: Sep. 7, 2016

Related U.S. Application Data
(60) Provisional application No. 61/953,984, filed on Mar. 17, 2014.

(51) Int. Cl. A61L 27/44; A61L 27/54; A61L 27/18; A61L 27/04; A61L 27/58; A61L 27/56

(52) U.S. Cl. CPC A61L 27/446; A61L 27/58; A61L 27/56; A61L 27/18; A61L 27/047; A61L 27/54; A61L 2300/604; A61L 2430/02; A61L 2430/34; A61L 2300/412; A61L 2300/102

(57) ABSTRACT

The invention relates to magnesium-polymer composites, methods for their preparation and applications for their use. The composites include a combination of magnesium particles and polymer matrix. The polymer can include, but is not limited to, poly(lactic co-glycolic) acid. In certain embodiments, the composites of the invention are particularly useful for forming medical devices for implantation into a body of a patient. In certain other embodiments, the magnesium-polymer composites are useful for wound healing compositions for administration to an exterior surface of a body of a patient.

### CROSS REFERENCE TO RELATED PATENT APPLICATION

This patent application claims the benefit of U.S. Provisional Patent Application No. 61/953,984, entitled “Magnesium/Polymer Composite-Containing Scaffolds to Enhance Tissue Regeneration”, filed on Mar. 17, 2014, the contents of which are incorporated herein by reference.

### GOVERNMENT SUPPORT AND FUNDING

The invention was made with government support under 0812348 awarded by the National Science Foundation (NSF). The government has certain rights in the invention.

### FIELD OF THE INVENTION

The invention relates to magnesium-polymer composites for use in wound healing. In particular, the magnesium-polymer composites are use in constructing medical devices, such as but not limited to scaffolds, for implantation into a body of a patient to enhance tissue regeneration and, more particularly, for orthopedic, periodontal, dental, craniofacial and cardiovascular applications. The magnesium-polymer composites of the invention are also suitable for use in other medical applications, such as but not limited to, magnesium-polymer compositions to be applied to an exterior surface of a body of a patient, such as skin, for wound healing. Furthermore, the polymer component of the magnesium-polymer composite is effective to provide sustained release of magnesium to the target area.

### BACKGROUND OF THE INVENTION

It is estimated that over 6.3 million bone fractures occur in the United States annually. Further, it is estimated that the medical cost associated with these injuries is approximately $14 billion. Fixation alone may be insufficient to regenerate large bone defects and non-unions. Treatment of these injuries may require hone grafts or the use of recombinant growth factor-containing scaffolds.

Implant devices, such as scaffolds, including but not limited to plates and screws, are commonly used in the practice of orthopedic, dental, craniofacial and cardiovascular implant surgery. In addition, stents are implanted into a body of a patient to support lumens, for example, coronary arteries. Furthermore, meshes and membranes are also used for guided tissue regeneration in various locations of the body to promote, e.g., favor, one tissue growth over another. Biomaterials for the construction of implant devices are typically chosen based on their ability to withstand cyclic load-hearing and their compatibility with the physiological environment of a human body. Many implant devices are traditionally constructed of polymer or metal. These materials of construction exhibit good biomechanical properties. Traditional metallic biomaterials, such as, titanium and stainless steel, in particular, have appropriate properties such as high strength, ductility, fracture toughness, hardness, corrosion resistance, formability, and biocompatibility to make them attractive for most load-bearing applications. For example, magnesium is attractive as a biomaterial because it is very lightweight, has a density similar to cortical bone, has an elastic modulus close to natural bone, is essential to human metabolism, is a cofactor for many enzymes, stabilizes the structures of DNA and RN and degrades safely m the body, Polymers, such as polyhydroxy acids, polylactic acid (PLA), polvglvcolic acid (PGA), and the like are known as conventional biomaterials, even though, in some instances, the strength and ductility exhibited by polymers is not as attractive as that demonstrated by metallic biomaterials.

With respect to biomaterials for medical implant devices, there has been an interest and focus to design and develop biodegradable construction materials such that the implant device is capable of degrading over a period of time, e.g., by dissolving in the physiological environment. Therefore, surgery is not required to remove the implant device when it is no longer needed. However, in some instances, there have been disadvantages associated with scaffolds constructed of biodegradable polymer, such as, but not limited to, the production of acidic degradation by-products, negative affect on protein and drug bioavailability in drug delivery applications, and exhibit of low mechanical strength and a lack of osteoconductivity.

In the field of biomedical applications, there is a desire to develop biocompatible materials of construction for scaffolds as medical implant devices wherein porous scaffolds are effective for bone regeneration and drug delivery In accordance with the invention, there is a desire to develop magnesium-polymer composites for scaffold construction which emphasize the beneficial properties of magnesium, such as osteoconductive and osteoinductive properties, and also de-emphasize the detrimental properties of the polymer, such as acidic by-products due to degradation. Further, it is desired to develop scaffolds and materials for their construction which improve delivery in a body of a patient, such as, but not limited to, magnesium, drugs, and bioactive agents. Furthermore, it is contemplated that the biocompatible materials of the invention are not limited to scaffold construction, but may also include magnesium-polymer compositions for use in wound healing.

### SUMMARY OF THE INVENTION

In one aspect, the invention provides a magnesium-polymer composite including magnesium particles and a polymer matrix, wherein the magnesium particles are embedded in the polymer matrix.

The magnesium particles can be selected from the group consisting, of pure magnesium particles and powder, magnesium alloy particles and powder, metallic magnesium, magnesium salt particles and powder, and combinations thereof.

The polymer matrix can be selected from the group consisting of calcium phosphate, hydroxyapatite, lecithin collagen, fibrin, gelatin, silk, elastin, chitosan, starch, alginate, hyaluronic acid, chondroitin, agarose, cellulose, polyester, poly(glycolic acid), poly(L-lactic acid), poly (lactic-co-glycolic acid), poly(caprolactone), poly(propylene fumarate), polyorthoester, polyanhydride, poly(etheylene glycol), polycarbonate, polyurethane, elastomer, poly(glycerol sebacate), and mixtures thereof.

At least one of the magnesium particles and the polymer matrix can be selected such that degradation rate is controllable. The concentration of at least one of the magnesium particles and the polymer can be selected such that pH is controllable. The concentration of the magnesium particles can be selected, such that said concentration is effective to buffer acidic by-products of degradation of the polymer matrix. Further, the purity of the magnesium particles is selected such that degradation rate is controllable. In certain embodiments, the magnesium particles include from about 99 to about 99.95 weight percent magnesium based on total weight of the particles.

In certain embodiments, the tissue is bone.

In another aspect, the invention provides a method of preparing a magnesium-polymer composite. The method includes selecting magnesium particles, selecting a polymer matrix, and embedding, the magnesium particles in the polymer matrix.

The magnesium particles can be selected from the group consisting of pure magnesium particles and powder, magnesium alloy particles and powder, metallic magnesium, magnesium salt particles and powder, and combinations thereof.

In still another aspect, the invention provides a medical implant device comprising the composite of claim 1. The medical implant device can be selected from the group consisting of plates, meshes, staples, screws, pins, tacks, rods, suture anchors, tubular mesh, coils, x-ray markers, catheters, endoprostheses, pipes, shields, bolts, clips or plugs, dental implants or devices, occlusive barrier membranes, graft devices, bone-fracture healing devices, bone replacement devices, joint replacement devices, tissue regeneration devices, cardiovascular stents, nerve guides, surgical implants and wires. The medical implant device can include a plurality of pores. The plurality of pores can be employed for drug delivery.

In certain embodiments, the polymer can contribute to the sustained delivery of the magnesium particles to an implant area in a body of a patient.

In yet another aspect, the invention provides a wound healing composition comprising the composite of claim 1.

### BRIEF DESCRIPTION OF THE DRAWINGS

A full understanding of the invention can be gained from the following, description of the preferred embodiments when read in conjunction with the accompanying drawings, in which:

FIGS. 1 and 2 show images of magnesium-polymer composites prepared in accordance with certain embodiments of the invention;

FIG. 3 shows stereo microscope images of samples of scaffolds wherein one sample was prepared in accordance with the prior art and three samples were prepared in accordance with certain embodiments of the invention;

FIG. 4 is a plot showing the pH of tissue culture medium in which scaffolds were placed, including scaffolds prepared in accordance with the prior art as compared to those prepared in accordance with certain embodiments of the invention;

FIG. 5 includes plots showing maximum strain and stress, modulus and porosity for scaffolds prepared in accordance with the prior art as compared to those prepared in accordance with certain embodiments of the invention;

FIG. 6 is a plot showing proliferation data for human hone marrow stromal cells cultured in tissue culture medium containing degraded scaffold extracts prepared in accordance with the prior art as compared to those prepared in accordance with certain embodiments of the invention;

FIG. 7 shows images of healed canine pre-molar tooth sockets following implantation of scaffolds prepared in accordance with certain embodiments of the invention; and

FIG. 8 is a plot showing the magnesium concentration release from Mg/PLGA scaffolds over time in accordance with certain embodiments of the invention.

### DETAILED DESCRIPTION OF THE PREFERRED EMBODIMENTS

The invention relates to novel, biocompatible magnesium-polymer composites, methods of preparing the biocompatible, magnesium-polymer composites, and articles that are constructed or fabricated of the biocompatible magnesium-polymer composites, In certain embodiments, the magnesium-polymer composites form articles, e.g., medical devices for implantation into a body, e.g., a human body, of a patient. In these embodiments, the articles are useful in medical applications, such as, but not limited to orthopedic, dental craniofacial and cardiovascular surgery. In other embodiments, the magnesium-polymer composites form articles for use on an exterior surface, such as, the skin of a body of a patient, for wound healing. In these embodiments, the magnesium-polymer composite can be in various forms, such as, but not limited to, a topical formulation, a bandage or patch.

Moreover, the magnesium-polymer composite in accordance with the invention is effective to provide a sustained release or delivery of magnesium to, for example, a wound site, or a bone or tissue environment. Without intending to be bound by any particular theory, it is believed that the release of magnesium, for example, into a bone environment is effective to trigger bone growth and/or regeneration. it is further believed that metal ions, such as, magnesium ions, contribute to the formation of bone. Thus, the polymer component in the magnesium polymer composite of the invention can he employed as a delivery system for magnesium, e.g., magnesium ions, into a bone environment.

For ease of description, portions of the disclosure herein are directed to scaffolds, in particular. However, it is understood that these portions of the disclosure are not intended to limit the scope of the invention and it is contemplated that the disclosure directed to scaffolds is, in fact, applicable to and encompasses other medical implant devices known in the art.

There are generally known polymers for use in producing scaffolds, however, they do riot provide the improvements and benefits demonstrated by the magnesium-containing polymer composites of the invention. For example, conventional polymers for use in Constructing scaffolds have been found to produce acidic by-products, which can cause inflammation in surrounding tissue and result in jeopardizing drug, gene and protein delivery capabilities of the scaffolds. In contrast, in accordance with the invention, the presence of magnesium in combination with polymer produces a scaffold, which exhibits a degradation profile that buffers polymer-related acidity and may ultimately improve the in-vio performance of polymer-based products. Further, the novel magnesium-containing polymer composites of the invention are effective for tissue regeneration and, in particular, bone regeneration, within a body of a patient.

The materials for use in the invention as the polymer component in the magnesium-polymer composite can be selected from a wide variety of natural and synthetic materials that are known in the art. Suitable materials include, but are not limited to calcium phosphate, hydroxyapatite, lecithin collagen, fibrin, gelatin, silk, elastin, chitosan, starch, alginate, hyaluronic acid, chondroitin, agarose, cellulose, polyester, such as poly(glycolic acid) (PGA), poly(L-lactic acid) (PLA), poly(lactic-co-glycolic acid) (PLGA), poly(caprolactone) (PCL), poly(propylene fumarate), polyorthoester, polyanhydride, poly(etheylene glycol) (PEG), polycarbonate, polyurethane, elastomer, such as but not limited to poly(glycerol sebacate) (PGS), and mixtures thereof.

In certain embodiments, wherein magnesium-polymer scaffolds are constructed to enhance tissue and/or bone regeneration or wound healing composites are prepared, the polymer component may be selected from calcium phosphate, collagen, fibrin, gelatin and mixtures thereof. In certain other embodiments, wherein the magnesium component of the magnesium-polymer composite has a buffering effect, e.g., provides for acidic degradation of by-products, the polymer component may be selected from poly(glycolic acid) (PGA), poly(L-lactic acid) (PLA), poly(lactic-co-glycolic acid) (PLGA), poly(caprolactone) (PCL), poly(propylene fumarate), and mixtures thereof.

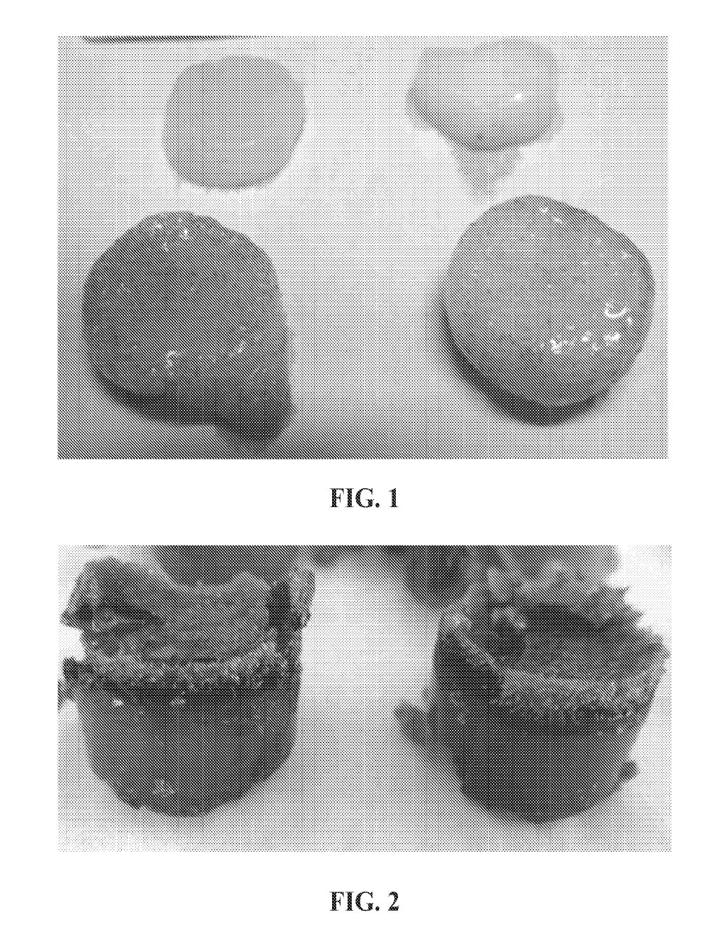

FIGS. 1 and 2 show images of magnesium-polymer composites prepared in accordance with certain embodiments of the invention. In particular, FIG. 1 shows a magnesium-polymer composite composed of magnesium and fibrin, and FIG. 2 shows a magnesium-polymer composite composed of magnesium, PEG and gelatin.

The selection of a particular polymer and its use in a specified amount or concentration, or range thereof, can provide the ability to control, customize and tailor the degradation rate of the polymer and therefore, the degradation rate of the scaffold that is constructed of the magnesium-polymer composite, which can enhance bone regeneration. In certain embodiments, varying the particular polymer and its specific concentration can provide a scaffold composite that is optimal for achieving bone regeneration. In certain embodiments, the magnesium-polymer composites include a combination of magnesium (Mg) powder or particles and poly(lactic co-glycolic) acid (PLGA).

Furthermore, the selection of particular magnesium particles having a specified size and purity, can provide the ability to control, customize and tailor the degradation rate of the polymer, which is combined with the magnesium particles, and therefore, the degradation rate of the scaffold that is constructed of the magnesium-polymer composite, which may enhance bone regeneration. The magnesium particles may be composed of pure magnesium or the magnesium particles may be composed of magnesium-containing alloy. In certain embodiments, the magnesium component in accordance with the invention is metallic magnesium, e.g., magnesium ions. In certain other embodiments, the purity of the magnesium particles can vary from about 99 to about 99.95 weight percent magnesium based on total weight of the particles. When the particles are composed of magnesium alloy, they can be selected from commercially available magnesium alloys, such as, but not limited to, AZ31 and WE43.

In certain embodiments, the magnesium-polymer composite is employed to construct scaffolds that exhibit a porous framework or configuration. The porous scaffolds can be manufactured using conventional apparatus and processes, such as, pressing, sintering and solvent casting with salt leaching. It is typical for conventional polymers to be limiting as to the complexity of scaffold geometries that may be formed. However, the magnesium-polymer composite of the invention is effective to form both simple geometries and complex geometries, which is advantageous when producing scaffolds for various applications and locations within a body.

Without intending to be bound by any particular theory, it is believed the combination of magnesium powder or particles, e.g., metallic magnesium, and polymer form a composite that exhibits improved mechanical properties and enhances bone regeneration associated with the scaffolds produced therefrom, as compared to scaffolds composed of only polymer. For example, conventional polymer meshes, membranes, fixation plates, screws and scaffolds are FDA approved and commercially available, but have poor mechanical properties and do not enhance bone regeneration. Current artificial bone scaffolds rely on recombinant human growth factors for enhanced bone healing which, is expensive, requires advanced manufacturing capabilities and, faces significant complication risks and regulatory scrutiny. Magnesium and magnesium alloys enhance bone growth and have received FDA IDE clearance for vascular stem applications. Magnesium and magnesium alloys have been shown in the literature to enhance cell proliferation, angiogenesis, bone regeneration and fracture healing. In certain embodiments, magnesium powder or particles can be added to existing FDA-approved polymers for synthesizing scaffolds demonstrating improved properties.

An important focus in tissue regeneration technology design and development is the ability to create patient and in 3D scaffolds. Magnesium powder or particles are not typically used in the art of 3D-printing primarily due to safety concerns relating to magnesium. However, in accordance with the invention, it is contemplated that embedding magnesium powder or particles within a polymer matrix may enable 3D printing of patient and injury-specific scaffolds containing magnesium, while alleviating safety concerns.

Non-limiting examples of medical implant devices in which the compositions and articles of the invention can be used include, but are not limited to plates, meshes, staples, screws, pins, tacks, rods, suture anchors, tubular mesh, coils, x-ray markers, catheters, endoprostheses, pipes, shields, bolts, clips or plugs, dental implants or devices, such as but not limited to occlusive barrier membranes, graft devices, bone-fracture healing devices, bone replacement devices, joint replacement devices, tissue regeneration devices, cardiovascular stems, nerve guides, surgical implants and wires. In a preferred embodiment, the medical devices include fixation bone plates and screws, temporomandibular joints, cardiovascular stents, and nerve guides.

The medical implant devices described herein can have at least one active substance attached thereto. The active substance can be either attached to the surface or encapsulated within the medical implant devices. As used herein, the term “active substance” describes a molecule, compound, complex adduct and/or composite that exhibits one or more beneficial activities such as therapeutic activity, diagnostic activity, biocompatibility, corrosion, and the like. Active substances that exhibit a therapeutic activity can include bioactive agents, pharmaceutically active agents, drugs and the like. Non-limiting examples of bioactive agents that can be incorporated in the composites, articles and devices of the invention include, but are not limited to, bone growth promoting agents such as growth factors, drugs, proteins, antibiotics, antibodies, ligands, DNA, RNA, peptides, enzymes, vitamins, cells and the like, and combinations thereof. Moreover, as previously described, herein, the magnesium-polymer composite in accordance with the invention is effect to provide a sustained and controlled release of magnesium to a physiological environment or target area of the body of the patient.

Further, in certain embodiments, other known components and additives may be included in the magnesium-polymer composites of the invention to impart additional characteristics and properties to the resulting scaffolds constructed therefrom, provided that the non-toxicity of the composites is maintained within acceptable limits. The additional components and additives can be selected from a wide variety known in the art and can include strontium, manganese, calcium zinc, rare earth elements, silver or any other element that may be included in the final alloy composition. For example, silver may be added to the magnesium-polymer composite to provide anti-microbial properties.

The magnesium-polymer composites of the invention can be prepared using various conventional methods and processes known in the art. In general, pressing, sintering and solvent casting with salt leaching methods can be employed. It is believed that the particular process used for casting may affect the properties and characteristics of the cast composite. In certain embodiments, the casting may be performed under a protective atmosphere to preclude, minimize or reduce decomposition of components in the composite, in particular, it is desirable to preclude, minimize or reduce decomposition of the magnesium in the composite. The protective atmosphere can include compounds selected from those known in the art, such as but not limited to, argon, sulfur hexafluoride and mixtures thereof. In further embodiments, the resulting cast can he subjected to various forming and finishing processes known in the art. Non-limiting examples of such processes include, but are not limited to extrusion, forging, polishing (by mechanical and/or chemical means), surface treating (to form a superficial layer on the surface) and combinations thereof.

In addition to forming a medical implant device, e.g., a scaffold, from the magnesium-polymer composite, in accordance with the invention, the magnesium-polymer composite can be deposited or applied to a substrate to form a film, layer or coating thereon. Various substrates known m the art can be used and can include, but are not limited to, non-resorbable and resorbable metals.

Further, the magnesium-polymer composites of the invention can be used for other wound healing applications, in addition to their uses relating to medical implant devices. That is, in accordance with the invention, the magnesium-polymer composites may provide enhanced wound healing in a wide variety of applications. In certain embodiments, magnesium powder or particles may be incorporated into other wound-healing polymers, such as, but not limited to, topically applied compositions to enhance healing of wounds on the exterior surface, e.g., skin, of a patient. The topically applied compositions can be in various forms known in the art, including lotions, gels, creams and the like. Suitable non-limiting examples include fibrin or collagen gels. The wounds on the surface of the skin can include a wide variety of skin conditions and lesions including, but not limited to, diabetic foot ulcers and pressure ulcers.

Additional objects, advantages and novel features of the invention may become apparent to one of ordinary skill in the art based on the following examples, which are provided for illustrative purposes and are not intended to be limiting.

### EXAMPLES

### Example 1

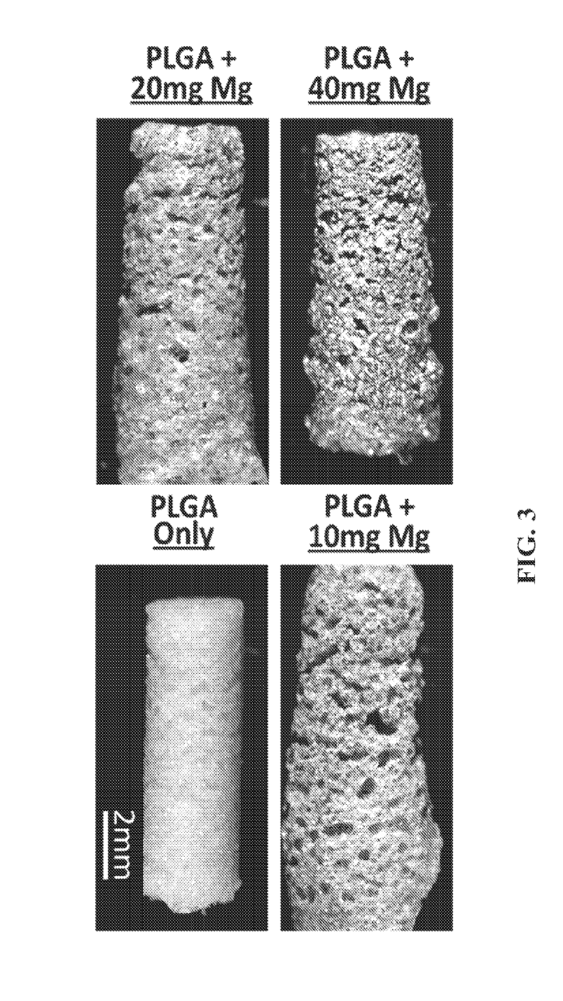

A first scaffold was prepared using 40 mg of PLGA, a second scaffold was prepared using a combination of PLGA and 10 mg of Mg powder, a third scaffold was prepared using a combination of PLGA and 20 mg of Mg powder and a fourth scaffold was prepared using a combination of PLGA and 40 mg of Mg powder. The Mg powder was embedded in the PLGA scaffold and varying amounts of porosity were added through a solvent casting/particulate leaching technique. FIG. 3 illustrates these scaffolds and demonstrates the improvement realized by the Mg-polymer composites as compared to the PLGA-only composite) through variation of porosity for tailored tissue regeneration properties.

### Example 2

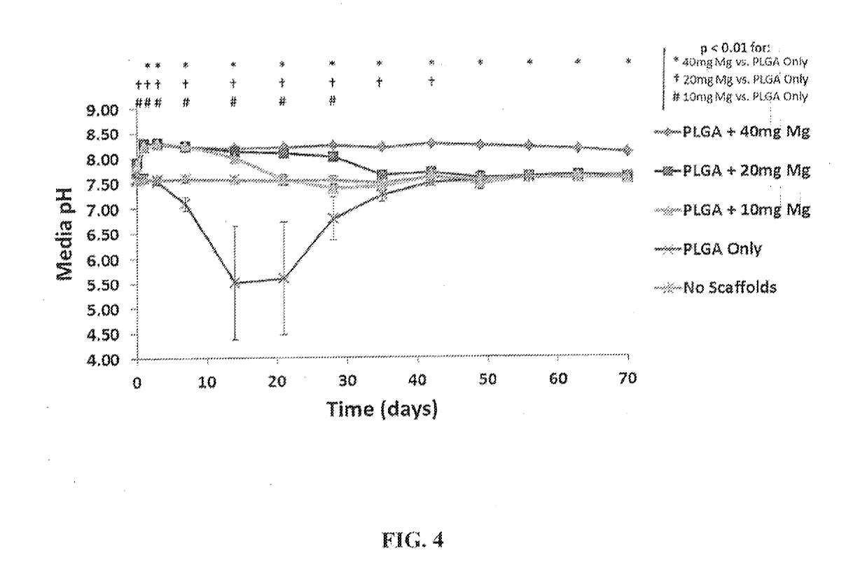

Scaffolds composed of only PLGA and scaffolds composed of a combination of Mg and PLGA were synthesized with varying amounts of Mg powder added, it was shown that degradation of PLGA-only scaffolds in tissue culture medium resulted in a highly acidic pH (that has been shown in the art to be detrimental to tissue regeneration and, drug and protein release in-vivo). The Mg-PLGA scaffolds demonstrated an ability to buffer the acidic degradation of PLGA and maintain tissue culture medium pH at a level that was not cytotoxic. These results were achieved with a Mg powder amount as low as 10 mg in the PLGA scaffold for 10 weeks. Further, it was demonstrated that increasing the amount of Mg powder, resulted in the ability to extend the release of Mg into the medium. FIG. 4 shows media pH data for PLGA scaffolds haying varying amounts of Mg incorporated therein, i.e., 0, 10 mg, 20 mg and 40 mg.

### Example 3

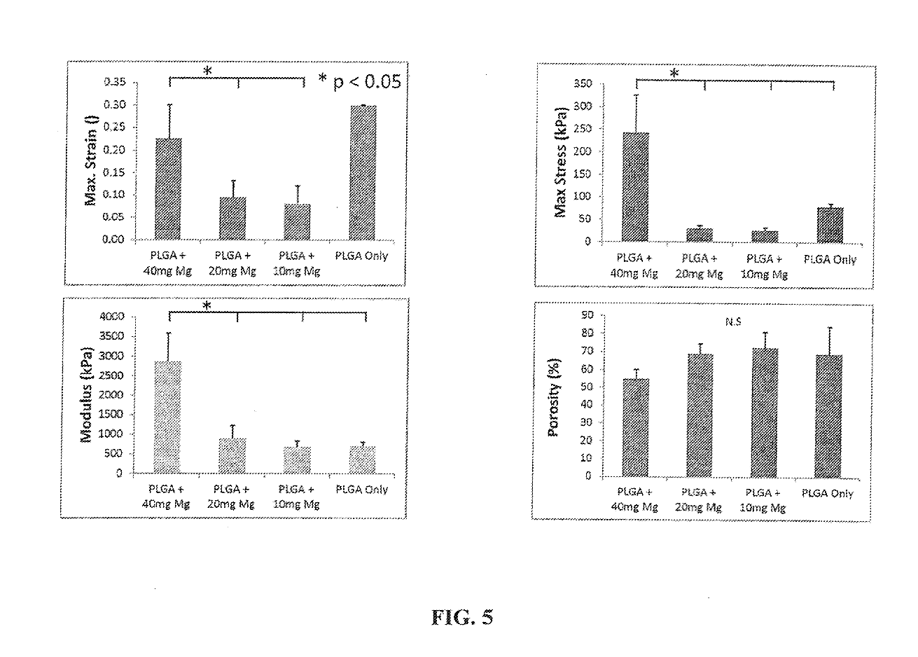

Scaffolds composed of only PLGA and scaffolds composed of a combination of Mg and PLGA Were synthesized with varying amounts of Mg powder added. It was shown that the mechanical properties of the Mg-PLGA scaffolds were improved as compared to the PLGA -only scaffolds. FIG. 5 shows plots of maximum strain and stress, modulus and porosity for PLGA scaffolds having varying amounts of Mg incorporated therein, i.e., 0, 10 mg, 20 mg and 40 mg. It was found that adding 40 mg of Mg powder to PLGA scaffolds increased both maximum stress and modulus as compared to PLGA scaffolds in the absence of Mg.

### Example 4

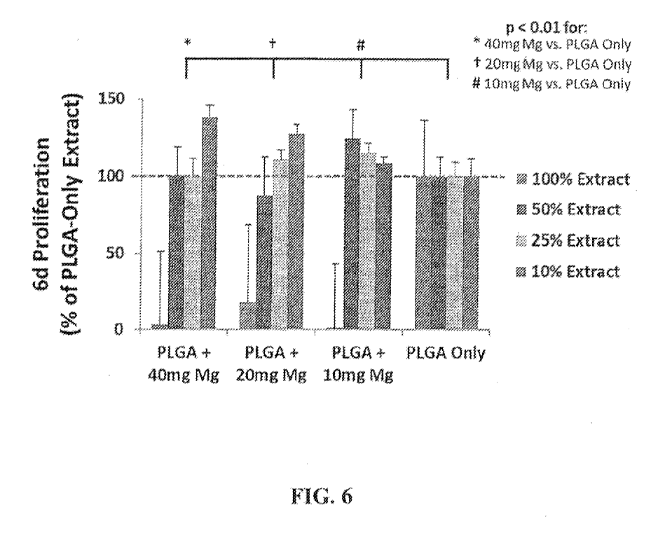

Scaffolds composed of only PLGA and scaffolds composed of a combination of Mg and PLGA were synthesized with varying amounts of Mg powder added. The proliferation of cells exposed to scaffold extracts was assessed. A standardized indirect crotoxicity assay was performed. It was found that while the proliferation of cells exposed to scaffold extracts was less than cells exposed to medium without extracts, the addition of Mg to PLGA scaffolds resulted in increased proliferation as compared to PLGA-only scaffolds. These results are illustrated in FIG. 6.

### Example 5

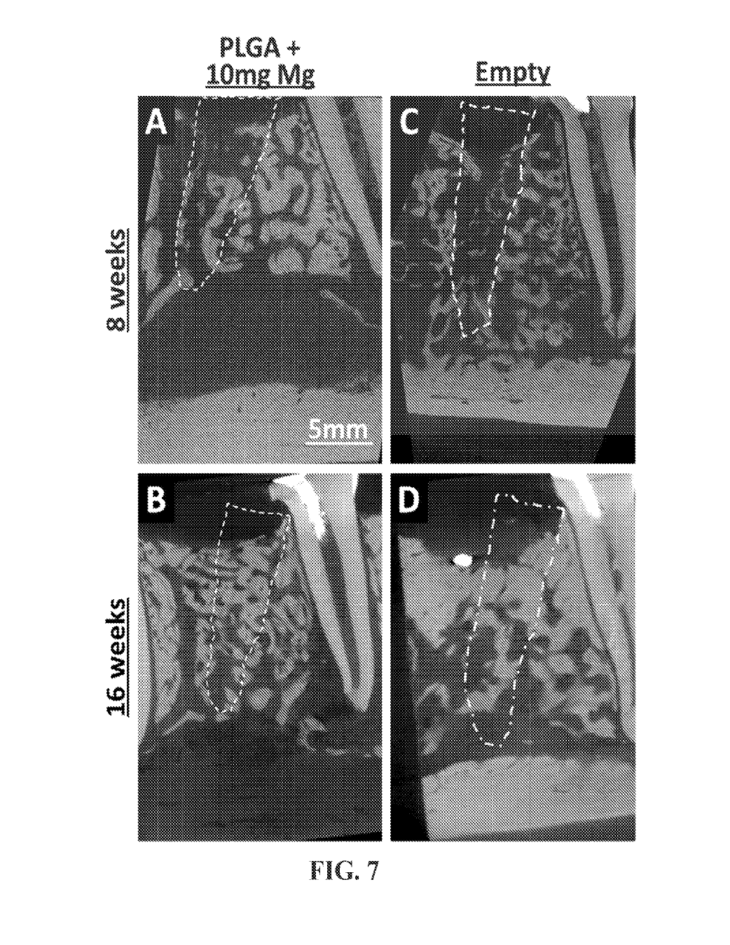

Scaffolds composed of a combination of PLGA and 10 mg of Mg powder were synthesized and implanted into canine tooth socket defects to assess in-vivo biocompatibility and suitability for dental socket preservation applications. The histology of 8-week explants showed bone formation throughout the defect with small remnants of Mg remaining. Additionally, a comparison to dog tooth root defects with no implants showed that the Mg-PLGA scaffolds may be capable of enhancing bone regeneration. These results are illustrated in FIG. 7, which show that implantation of PLGA and 10 mg of Mg scaffolds into canine pre-molar tooth sockets increased the bone height as compared with empty defects. Views A and B show the PLGA and 10 mg of Mg at 8 weeks and 16 weeks, respectively. Views C and D show empty defects.

### Example 6

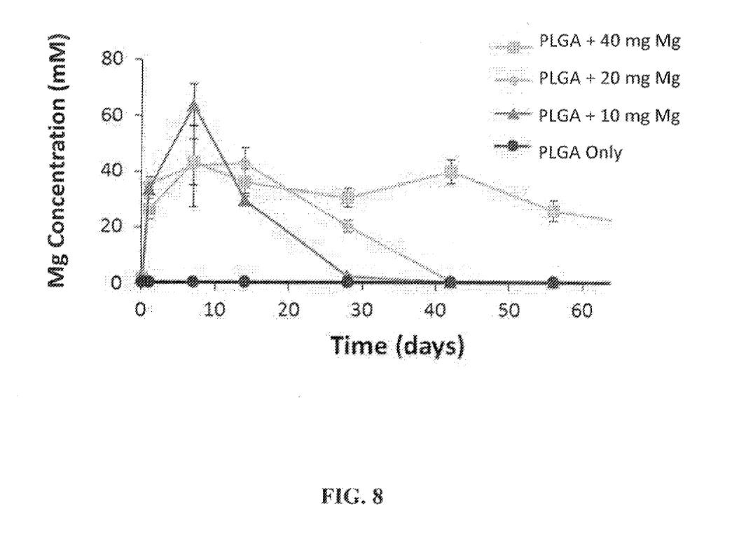

Scaffolds composed of only PLGA and scaffolds composed of a combination of Mg and PLGA were synthesized with varying amounts of Mg powder added. The Mg/PLGA scaffolds were cultured in tissue culture medium with FBS that was replaced weekly. Media samples were subjected to inductively coupled plasma atomic emission spectroscopy to quantify the concentration of magnesium release. The addition of Mg particles to PLGA scaffolds enabled the controlled release of magnesium into the surrounding environment, with increasing amounts of magnesium added resulting in longer release times. These results are illustrated in FIG. 8.

We claim:

1. A magnesium-polymer composite for tissue healing and regeneration, comprising:
magnesium particles; and
polymer matrix, wherein the magnesium particles are embedded in the polymer matrix.

2. The composite of claim 1, wherein the magnesium particles are selected from the group consisting of pure magnesium particles and powder, magnesium alloy particles and powder, metallic magnesium, magnesium salt particles and powder, and combinations thereof.

3. The composite of claim 1, wherein the polymer matrix is selected from the group consisting of calcium phosphate, hydroxyapatite, lecithin, collagen, fibrin, gelatin, silk, elastin, chitosan, starch, alginate, hyaluronic acid, chondroitin, agarose, cellulose, polyester, poly(glycolic acid), poly(L-lactic acid), poly(lactic-co-glycolic acid), poly(caprolactone), poly(propylene fumarate), polyorthoester, polyanhydride, poly(etheylene glycol), polycarbonate, polyurethane, elastomer, poly(glycerol sebacate), and mixtures thereof.

4. The composite of claim 1, wherein at least one of the magnesium particles and the polymer matrix is selected such that degradation rate is controllable.

5. The composite of claim 1, wherein concentration of at least one of the magnesium particles and the polymer is selected such that pH is controllable.

6. The composite of claim 1, wherein concentration of the magnesium particles is selected such that said concentration is effective to buffer acidic by-products of degradation of the polymer matrix.

7. The composite of claim 1, wherein purity of the magnesium particles is selected such that degradation rate is controllable.

8. The composite of claim 7, wherein the magnesium particles comprise from about 99 to about 99.95 weight percent magnesium based on total weight of the particles.

9. The composite of claim 1, wherein the tissue is bone.

10. A method of preparing a magnesium-polymer composite, comprising:
selecting magnesium particles;
selecting a polymer matrix; and
embedding the magnesium panicles in the polymer matrix.

11. The method of claim 10, wherein the magnesium particles are selected from the group consisting of pure magnesium particles and powder, magnesium alloy panicles and powder, metallic magnesium, magnesium salt particles and powder, and combinations thereof.

12. A medical implant device comprising the composite of claim 1.

13. The medical implant device of claim 12, wherein said device is selected from the group consisting of plates, meshes, staples, screws, pins, tacks, rods, suture anchors, tubular mesh, coils, x-ray markers, catheters, endoprostheses, pipes, shields, bolts, clips or plugs, dental implants or devices, occlusive barrier membranes, graft devices, bone-fracture healing devices, bone replacement devices, joint replacement devices, tissue regeneration devices, cardiovascular stents, nerve guides, surgical implants and wires.

14. The medical implant device of claim 12, comprising a plurality of pores.

15. The medical implant device of claim 14, wherein the plurality of pores are employed for drug delivery.

16. The medical implant device of claim 12, wherein the polymer contributes to the sustained, delivery of the magnesium particles to an implant area in a body of a patient.

17. A wound healing composition comprising the composite of claim 1.
